(19) United States
(12) Patent Application Publication
(10) Pub. No.: US 2022/0409110 A1
(43) Pub. Date: Dec. 29, 2022

(54) INFERRING COGNITIVE LOAD BASED ON GAIT

(71) Applicant: Hewlett-Packard Development Company, L.P., Spring, TX (US)

(72) Inventors: Nataliya Rokhmanova, Palo Alto, CA (US); Sarthak Ghosh, Palo Alto, CA (US); Rafael Ballagas, Palo Alto, CA (US); Mithra Vankipuram, Palo Alto, CA (US)

(73) Assignee: Hewlett-Packard Development Company, L.P., Spring, TX (US)

(21) Appl. No.: 17/773,099

(22) PCT Filed: Nov. 12, 2019
(86) PCT No.: PCT/US2019/060875

§ 371 Date: Apr. 29, 2022

(51) Int. Cl. A61B 5/16; G06F 3/01; G06F 3/14; A61B 5/11; A61B 5/00

(52) U.S. Cl. CPC A61B 5/165; G06F 3/012; G06F 3/14; A61B 5/112; A61B 5/1114; A61B 5/7278; A61B 5/6803; A61B 5/7267; A61B 5/0024; A61B 5/7445

(57) ABSTRACT

In various examples, a cognitive load of a user may be inferred. Motion sensor data indicative of head movement of the user may be generated with a motion sensor disposed adjacent a head of the user. The motion sensor data may be analyzed to infer a feature of a gait of the user. The user's cognitive load may be inferred based on the feature of the gait.

### BACKGROUND

With some types of immersive computing, an individual wears a head-mounted display (“HMD”) in order to have an augmented reality (“AR”) and/or virtual reality (“VR”) experience. As the popularity of the head-mounted display (“HM”) increases, its potential use cases are expanding as well. One such use case could be inferring cognitive load of a wearer of the HMD. Inferring cognitive load may have a variety of applications, such as wayfinding in an unfamiliar environment, immersive training in workplaces such as factories or plants, skill maintenance in professional fields such as medicine and dentistry, telepresence operation, and so forth.

### BRIEF DESCRIPTION OF THE DRAWINGS

Features of the present disclosure are illustrated by way of example and not limited in the following figure(s), in which like numerals indicate like elements.

FIG. 1 depicts an example environment in which selected aspects of the present disclosure may be implemented.

FIG. 2 demonstrates an example of how data may be processed from acquisition of head movement data to inferences of gait feature and cognitive load, and, ultimately, to application of cognitive load in a downstream process.

FIG. 3 depicts examples of remote computing devices that may receive, infer, and/or apply a user's cognitive load for a variety of purposes.

FIG. 4 depicts an example method for practicing selected aspects of the present disclosure.

FIG. 5 depicts an example method for practicing selected aspects of the present disclosure.

FIG. 6 shows a schematic representation of a computing device, according to an example of the present disclosure.

### DETAILED DESCRIPTION

For simplicity and illustrative purposes, the present disclosure is described by referring mainly to an example thereof. In the following description, numerous specific details are set forth in order to provide a thorough understanding of the present disclosure. It will be readily apparent however, that the present disclosure may be practiced without limitation to these specific details. In other instances, some methods and structures have not been described in detail so as not to unnecessarily obscure the present disclosure.

Additionally, it should be understood that the elements depicted in the accompanying figures may include additional components and that some of the components described in those figures may be removed and/or modified without departing from scopes of the elements disclosed herein. It should also be understood that the elements depicted in the figures may not be drawn to scale and thus, the elements may have different sizes and/or configurations other than as shown in the figures.

Various correlations has been demonstrated between feature(s) of an individual's gait and the individual's cognitive load. For example, it has been observed that some people tend to walk more quickly and/or take longer strides when not concentrating heavily. By contrast, some people tend to walk more slowly and/or take shorter strides when under a heavier cognitive load, and in some cases, their gaits may be wider.

Techniques are described herein for inferring an individual's cognitive load based on feature(s) of the individual's gait. These gait feature(s) may themselves be inferred using motion data generated by a motion sensor deployed adjacent the individual's head, e.g., integral with or otherwise part of a HMD. This motion sensor may take various forms, such as various types of accelerometers, a piezoelectric sensor, a gyroscope, a magnetometer, a gravity sensor, a linear acceleration sensor, and so forth, The inferred gait features—which may include foot plants on the ground or “strikes,” stride length, stride width, etc.—may then be used, alone or in combination with a variety of other signals, to infer a cognitive load of the individual.

In various examples, a classifier or machine learning model (these terms will be used interchangeably herein) may be trained to map head movement of a user to feature(s) of the user's gait. Put another way, the classifier/machine learning model may be trained to generate, based on the motion data generated by the motion sensor adjacent the individual's head, output that infers feature(s) of a gait of an individual. These classifiers/machine learning models may take a variety of different forms, including but not limited to a support vector machine, a random forest, a decision tree, various types of neural networks, a recurrent neural network such as a long short-term memory (“LSTM”) network or gated recurrent unit (“GRU”) network, etc.

To train the classifier/machine learning model, in some examples, other sensors, such as position trackers/motion sensors, may be deployed at other locations on an individual, such as adjacent their feet, to obtain data about motion of the individual's feet. A correlation or mapping may then be identified between a motion component of a foot of the individual and a motion component of the head of the individual determined from the motion sensor deployed adjacent the individual's head. The classifier may be trained based on and/or to include the correlation/mapping,

In some examples, the motion component of the individual's foot is a vertical displacement. For example, a period of time in which there is no change in a vertical displacement signal (e.g., y component of a signal generated by a three-axis accelerometer) generated by a foot-mounted motion sensor may correspond to a planted foot of the individual. Additional motion components may also be available directly or indirectly from the sensor disposed on the individual's foot. For example, a stride length (x component) or stride width (z component) may be calculated in some examples by integrating a signal from a 3-axis accelerometer. Alternatively, in some examples, positional information may be obtained by converting a motion sensor signal to quaternion forms or Euler angles relative to a ground or origin frame of reference. In some examples, walking speed may be calculated as stride length divided by time between successive foot plants of the same foot.

In some examples, the motion component of the individual's head is also a vertical displacement, e.g., a y component of a signal generated by a three-axis accelerometer installed in or integral with a HMD. Accordingly, in some examples, the vertical displacement signal generated by the foot-mounted motion sensor may be correlated/mapped to the vertical displacement signal generated by the head-mounted motion sensor. For example, one of the aforementioned classifiers or machine learning models may be trained to predict or infer foot plants or “strikes” based on head position.

Once this classifier is trained, it may be used to analyze the wearer's head position in order to infer gait features of the wearer, such as foot plants, stride length, stride width, walking speed, etc. These inferred gait features may then be analyzed in concert with other signals order to infer the wearer's cognitive load. In some examples, this cognitive load prediction may be performed using another classifier or machine learning model, referred to herein as a “cognitive load classifier.” In some such examples, the cognitive load classifier may be further trained using these predictions in conjunction with ground truth data the wearer self-reports about his or her perceived cognitive load. For example, the self-reported ground truth data may be used to train the classifier using techniques such as back propagation and/or gradient descent.

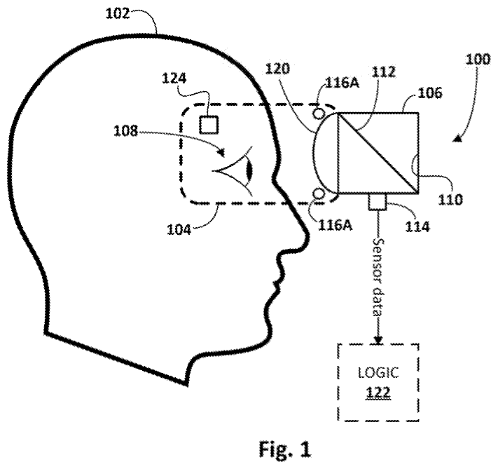

Referring now to FIG. 1, an example head-mounted display (“HMD”) 100 configured with selected aspects of the present disclosure is depicted schematically as it might be worn by an individual 102, which in the present context may also be referred to as a “user” or “wearer.” In FIG. 1, HMD 100 includes a first housing 104 and a second housing 106. However, in other examples, other housing configurations may be provided. First housing 104 encloses, among other things, an eye 108 of individual 102, which in this case is the individual's right eye. Although not visible in FIG. 1 due to the viewing angle, in many examples, first housing 104 may also enclose another eye of individual 102, which in this case would be the individual's left eye.

Second housing 106 may include some or all of the circuitry of HMD 100 that operate to provide individual 102 with an immersive computing experience. For example, in FIG. 1, second housing 106 includes a display 110, which in many cases may include two displays, one for each eye 108 of individual 102, that collectively render content in stereo. By rendering virtual content on display 110, HMD 100 provides individual 102 with a VR-based immersive computing experience in which individual 102 may interact with virtual objects, e.g., using his or her gaze. In some such examples, first housing 104 may completely enclose the eyes of individual 102, e.g., using a “skirt” or “face gasket” of rubber, synthetic rubber, silicone, or other similar materials, in order to prevent outside light from interfering with the individual's VR experience.

In some examples, HMD 100 may provide individual 102 with an AR-based immersive computing experience. For example, display 110 may be transparent so that individual 102 may see the physical world beyond display 110. Meanwhile, display 110 may be used to render virtual content, such as visual annotations of real world objects sensed by an external camera (not depicted) of HMD 100. In some such examples, HMD 100 may take the form of a pair of “smart glasses” with a relatively compact and/or light form factor. In some such examples, various components of FIG. 1 may be omitted, sized differently, and/or arranged differently to accommodate the relatively small and/or light form factor of smart glasses.

In some examples, including that of FIG. 1, second housing 106 includes a mirror 112 that is angled relative to second housing 106. Mirror 112 is tilted so that a field of view (“FOV”) of a vision sensor 114 is able to capture eye 108 of individual 102, Light sources 116A and 116E are also provided, e.g., in first housing 104, and may be operated to emit light that is reflected from eye 108 to mirror 112, which redirects the light towards vision sensor 114.

Vision sensor 114 may take various forms. In some examples, vision sensor 114 may be an infrared (“IR”) camera that detects electromagnetic radiation between 400 nm to 1 mm, or, in terms of frequency, from approximately 430 THz to 300 GHz. In some such examples, light sources 116 may take the form of IR light-emitting diodes (“LED”). Additionally, mirror 112 may be specially designed to allow non-IR light to pass through, such that content rendered on display 110 is visible to eye 108, while IR light is reflected towards vision sensor 114. For instance, mirror 112 may take the form of a dielectric mirror, e.g., Bragg mirror. In some examples, mirror 112 may be coated with various materials to facilitate IR reflection, such as silver or gold. In other examples, vision sensor 114 (and light source 116A/B) may operate in other spectrums, such as the visible spectrum, in which case vision sensor 114 could be an RGB camera.

The example of FIG. 1 is not meant to be limiting, and vision sensor 114, or multiple vision sensors, may be deployed elsewhere on or within HMD 100. In some examples, various optics 120 may be provided, e.g., at an interface between first housing 104 and second housing 106. Optics 120 may serve various purposes and therefore may take various forms. In some examples, display 110 may be relatively small, and optics 120 may serve to magnify display 110, e.g., as a magnifying lens. In some examples, 120 optics may take the form of a Fresnel lens, which may be lighter, more compact, and/or most cost-effective than a non-Fresnel magnifying lens. Using a Fresnel lens may enable first housing 104 and/or second housing 106 to be manufactured into a smaller form factor.

HMD 100 may facilitate eye tracking in various ways. In some examples, light sources 116A-B may emit coherent and/or incoherent light into first housing 104. This emitted light may reflect from eye 108 in various directions, including towards mirror 112. As explained previously, mirror 112 may be designed to allow light emitted outside of the spectrum of light sources 116A-B to pass through, and may reflect light emitted within the spectrum of light sources 116A-B towards vision sensor 114. Vision sensor 114 may capture vision data that is then provided (as part of sensor data) to logic 122. Logic 122 may be integral with, or remote from, HMD 100. Vision data may take the form of, for example, a sequence of images captured by vision sensor 114. Logic 122 may perform various types of image processing on these images to determine various aspects of eye 108, such as its pose (or orientation), pupil dilation, pupil orientation, a measure of eye openness, etc.

Logic 122 may take various forms. In some examples, logic 122 may be integral with HMD 100, and may take the form of a processor (or multiple processors) that executes instructions stored in memory (not depicted). For example, logic 122 could include a central processing unit (“CPU”) and/or a graphics processing unit (“GPU”). In some examples, logic 122 may include an application specific integrated circuit (“ASIC”), a field-programmable gate array (“FPGA”), and/or other types of circuitry that perform selected aspects of the present disclosure. In this manner, logic 122 may be circuitry or a combination of circuitry and executable instructions,

In other examples, logic 122 may not be integral with HMD 100, or may be implemented across multiple devices, including or not including HMD 100. In some examples, logic 122 may be partially or wholly implemented on another device operated by individual 102, such as a smart phone, smart watch, laptop computer, desktop computer, set top box, a remote server forming part of what may be referred to as the “cloud,” and so forth. For example, logic 122 may include a processor of a smart phone carried by individual 102. Individual 102 may operably couple the smart phone with HMD 100 using various wired or wireless technologies, such as universal serial bus (“USB”), wireless local area networks (“LAN”) that employ technologies such as the Institute of Electrical and Electronics Engineers (“IEEE”) 802.11 standards, personal area networks, mesh networks, high-definition multimedia interface (“HDMI”), and so forth. Once operably coupled, individual 102 may wear HMD 100, which may render content that is generated by the smart phone on display 110 of HMD 100. For example, individual 102 could install a VR-capable game on the smart phone, operably couple the smart phone with HMD 100, and play the VR-capable game through HMD 100.

In some examples, HMD 100 may include a motion sensor 124 that generates a signal indicative of detected head movement of individual 102. In some examples, motion sensor 124 may generate a signal that is usable to infer, directly or indirectly, components in one, two, or even three dimensions, These components may include: vertical displacement, which is described herein as a change along the y axis; horizontal displacement in a direction of the individual's walk, which is described herein as a change along the x axis; and lateral displacement, which is described herein as a change along the z axis. Motion sensor 124 may take various forms, such as a three-axis accelerometer, other types of accelerometers, a gyroscope, a piezoelectric sensor, a gravity sensor, a magnetometer, and so forth.

While motion sensor 124 is depicted in a particular location of HMD 100 in FIG. 1, this is not meant to be limiting. Motion sensor 124 may be located on or with HMD 100 at any number of locations. In some examples, motion sensor 124 may be installed within HMD 100, e.g., during manufacturing, so that it is not easily accessible. In other examples, motion sensor 124 may be a modular component that can be removably installed on or within HMD 100.

Motion sensor 124 may be operably coupled to logic 122 via any of the aforementioned technologies. Accordingly, in various examples, logic 122 may analyze a motion signal it receives from motion sensor 124. Based on this analysis, logic 122 may infer a feature of a gait of individual 102. This feature of the gait of individual 102 may then be used, e.g., by logic 122 or by separate logic implemented elsewhere, to infer a cognitive load of individual 102.

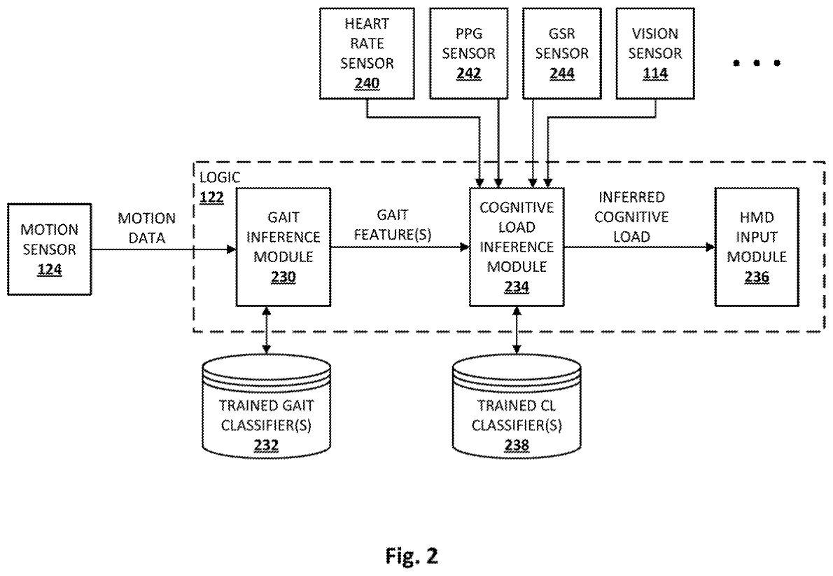

FIG. 2 schematically depicts one example of how data may be processed using techniques described herein to infer and apply a cognitive load of a user. Motion sensor 124 provides the motion data it generates to logic 122 as described previously. As also noted previously, logic 122 may be hosted wholly or partially onboard HMD 100, on another computing device operated by individual 102, such as a smart phone, or remotely from individual 102 and HMD 100.

Logic 122 may operate various modules using any combination of circuitry or combination of circuitry and machine-executable instructions. For example, in FIG. 2, logic 122 operates a gait inference module 230, a cognitive load inference module 234, and an HMD input module 236. In other examples, any one of modules 230-236 may be combined with other modules and/or omitted.

Gait nference module 230 applies all or selected parts of motion data received from motion sensor 124 as input across a trained classifier obtained from a trained gait classifier database 232 to generate output. The output generated based on the classifier may indicate, or may be used to calculate, various features of a gait of the user, such stride length, foot plant timing, stride width, walking speed, and so forth.

Gait classifier database 232 may be maintained in whole or in part in memory of HMD 100. For example, classifier(s) in database 232 may be stored in non-volatile memory of HMD 100 until needed, at which point they may be loaded into volatile memory. As used herein, memory may refer to any electronic, magnetic, optical, or other physical storage device that stores digital data. Volatile memory, for instance, may include random access memory (“RAM”). More generally, memory may also take the form of electrically-erasable programmable read-only memory (“EEPROM”), a storage drive, an optical drive, and the like.

Various types of classifiers and/or machine learning models may be trained and used, e.g., by gait inference module 230, to make infer various features of a user's gait. In some examples, the classifier or machine learning model may take the form of a support vector machine, a random forest, a decision tree, various types of neural networks such as a convolutional neural network (“CNN”), a recurrent neural network, an LSTM network, a GRU network, multiple machine learning models incorporated into an ensemble model, and so forth. In some examples, a classifier/machine learning model may learn weights in a training stage utilizing various machine learning techniques as appropriate to the classification task, which may include, for example linear regression, logistic regression, linear discriminant analysis, principal component analysis, classification trees, naive Bayes, k-nearest neighbors, learning vector quantization, support vector machines, bagging forests, random forests, boosting, AdaBoost, etc.

The gait feature(s) contained in the output generated by gait inference module 230 may be received by cognitive load inference module 234. Cognitive load inference module may apply the gait feature(s), e.g., in concert with other signals, as input across a trained cognitive load classifier (or machine learning model) obtained from a trained cognitive load (“CL” in FIG. 2) classifier(s) database 238. Based on this application, cognitive load inference module 234 may generate output indicative of an inferred cognitive load of the user. This inferred cognitive load may be received in some examples by a HMD input module 236.

As shown in FIG. 2, cognitive load inference module 234 may infer a cognitive load using signals other than gait feature(s) provided by gait inference module 230. In various examples, these other signals may include physiological signals, e.g., generated by physiological sensors, vision data, calendar data (e.g., is the user scheduled to be taking a test or studying?), social network status updates (“I'm so overworked!”), number of applications open on a computing device operated by the user, and so forth.

In FIG. 2, the additional signals include a heart rate signal generated by a heart rate sensor 240, a blood flow (or heart rate) signal generated by a photoplethysmogram (“PPG”) sensor 242, a galvanic skin response (“GSR”) signal generated by a GSR sensor 244, and vision data generated by the aforementioned vision sensor 114. As indicated by the ellipsis to the right, other sensors may be provided in addition to or instead of those depicted in FIG. 2, such as a thermometer, glucose meter, sweat meter, and so forth, And the particular combination of sensors in FIG. 2 is not meant to be limiting. Various sensors may be added or omitted.

Heart rate sensor 240 may take various forms, such as an electrocardiogram (“ECG”) sensor, a PPG sensor (in which case a separate PPG sensor 242 would not likely be included), and so forth. GSR sensor 244, which may also be referred to as an electrodermal activity (“EDA”) sensor, may take the form of, for example, an electrode coupled to the user's skin. Vision sensor 114 was described previously, and may provide vision data that includes features of the user's eye that may be used, in addition to eye tracking, for inferring cognitive load. These features of the user's eye may include, for instance, a measure of pupil dilation that is sometimes referred to as “pupillometry,” any measure of eye movement that may suggest heightened (or decreased) concentration, or any other eye feature that may indicative heightened or decreased cognitive load.

Some non-gait-related inputs to cognitive load inference module 234, such as PPG and galvanic skin response, are sensitive to motion, and thus may become noisy if the user is moving (e.g., walking). Other inputs, such as pupillometry data and gait features, are less sensitive to movement. Accordingly, in some examples, cognitive load inference module 234 may weigh various inputs differently depending on whether the user is determined to be moving. For example, cognitive load inference module 234 may assign less weight to movement-sensitive signals like PPG and/or galvanic skin response—and in some cases may assign greater weight to movement-insensitive signals like pupillometry—based on presence and/or magnitude of a gait feature provided by gait inference module 230.

HMD input module 236 may provide the inferred cognitive load to any number of applications, whether executing onboard HMD 100, onboard a mobile phone operably coupled with HMD 100, or on a remote computing system, such as servers(s) that are sometimes collectively referred to as a “cloud.” These applications may take various actions based on the inferred cognitive load.

A user concentrating heavily on a task at hand, and thereby operating under a heavy cognitive load, may be otherwise distracted from their surroundings. Accordingly, various actions may be taken to assist such a distracted user. Suppose HMD 100 is an AR device that allows a user to see their physical surroundings. Suppose further that the user is attempting to navigate through an unfamiliar environment, and consequently, the user is operating under a heavy cognitive load that is inferred using techniques described herein.

To ensure the user doesn't collide with an object in the user's path, logic 122 of HMD 100 may render, on display 110 of HMD 100, a visually emphasizing graphical element that overlays or is otherwise adjacent to a real-world object in the user's path. For example, logic 122 may render a graphical annotation (words and/or images) to look out for the object, may highlight, color, or otherwise render animation on or near the object to make it more conspicuous, etc.

Alternatively, in a similar scenario, a mapping application operated by logic 122 may receive the inferred cognitive load from HMD input module 236 or directly from cognitive load inference module 234. If the inferred cognitive load satisfies some threshold, the mapping application may visually emphasize points of interest and/or reference to the user on display 110 to decrease a likelihood that the user will miss a turn or get lost. By contrast, if the inferred cognitive load fails to satisfy the threshold, the mapping application may reduce or eliminate visual aid rendered to the user.

In some examples, the inferred cognitive load may be used to prioritize applications and/or notifications provided by applications, e.g., to avoid distracting the user and/or to help the user concentrate on the task at hand. For example, logic 122 of HMD 100 may operate a plurality of applications at once, as occurs frequently on many computing systems. One application may be the focus of the user, and therefore runs in the “foreground,” which means any inputs by the user are most likely directed to that foreground application. Other applications may run in the background, which means they are not currently being actively engaged with by the user,

Background applications may nonetheless provide notifications to the user, e.g., as cards or pop-up windows rendered on display and/or as audible notifications such as sound effects, natural language output (“Someone has been spotted at your front door”), etc. These notifications may be distracting to a user operating under a heavy cognitive load. Accordingly, in some examples, in response to a relatively heavy inferred cognitive load, HMD input module 236 may block, visually diminish, or otherwise demote notifications and/or other activity generated by background applications so that the user can focus more on the foreground activity.

In FIG. 2, the inferred cognitive load is used by HMD input module 236 to effect applications and/or other activity onboard HMD 100. However, this is not meant to be limiting. In various implementations, the inferred cognitive load may trigger a response on other computing devices, such as a smart watch, mobile phone, or any other computing device in wired or wireless communication with HMD 100. Additionally, in some implementations, gait inference module 230 may be implemented on one device, such as HMD 100, and cognitive load inference module 234 may be implemented elsewhere, e.g., on a mobile phone, tablet computer, smart watch, laptop, or other computing device operated by the user.

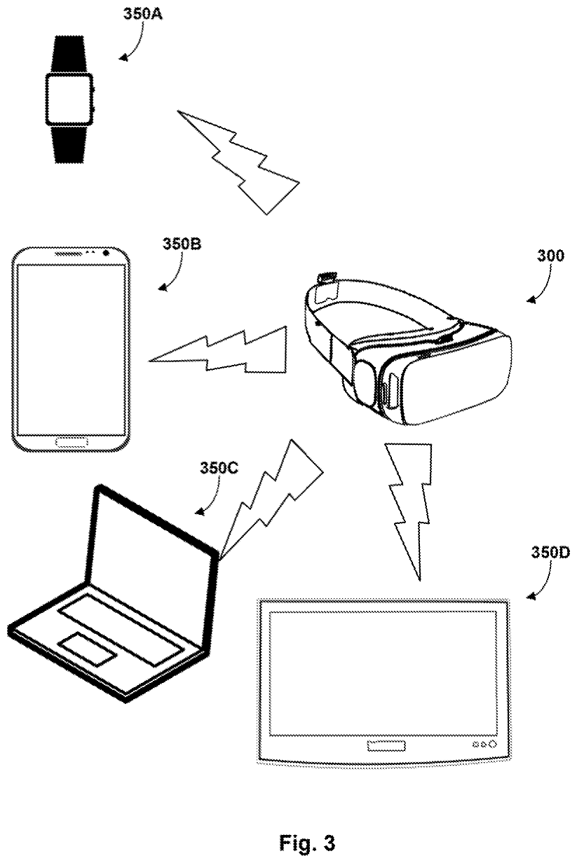

Referring now to FIG. 3, in some examples, a HMD 300 configured with selected aspects of the present disclosure may wirelessly transmit inferred gait feature(s) and/or an inferred cognitive load of a user wearing HMD 300 to various types of computing devices 350A-D for various purposes. These computing devices 350A-D may receive the gait feature(s) and infer the cognitive load themselves, or they may receive the cognitive load as already inferred by HMD 300.

Different types of computing devices may use cognitive load for different purposes. Mobile computing devices such as a smart watch 350A and mobile phone 350B may be carried/worn by the user while the user walks and wears HMD 300. These mobile computing devices 350A-B may reprioritize notifications and/or other application activity, e.g., as described previously, to avoid distracting the user and/or to allow the user to focus on a particular task, such as navigating through an unfamiliar environment, playing an AR mobile game, searching for a particular person, place, or thing, and so forth.

Other, less mobile devices 3500-D may also take various actions based on a cognitive load that they infer themselves or receive from HMD 300. Although not as mobile as computing devices 350A-B, computing devices 3500-D may be used by an ambulatory user in situations in which the user walks or otherwise exercises without necessarily changing locations, such as when the user is exercising on a treadmill. For example, the user may operate a laptop computer 350C to play music while the user exercises, or the user may operate a smart television 350D to play content the user watches while they exercise. Suppose the user receives a telephone call while exercising, which increases the user's inferred cognitive load. One or both computing devices 3500-D may take various responsive actions to allow the user to focus on the telephone call, such as turning down the volume, pausing playback, etc.

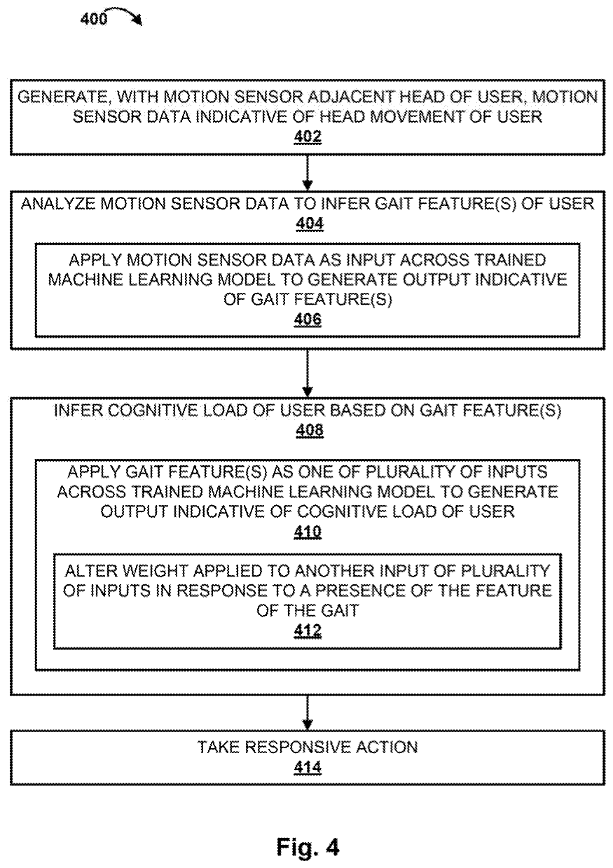

FIG. 4 illustrates a flowchart of an example method 400 for inferring a feature of a gait and from that, inferring a cognitive load. Some operations of FIG. 4 may be performed by a processor, such as a processor of the various computing devices/systems described herein, including logic 122. For convenience, operations of method 400 will be described as being performed by a system configured with selected aspects of the present disclosure. Other implementations may include additional operations than those illustrated in FIG. 4, may perform operations(s) of FIG. 4 in a different order and/or in parallel, and/or may omit various operations of FIG. 4.

At block 402, the system may generate, with a motion sensor disposed adjacent a head of the user, motion sensor data indicative of head movement of the user. As noted previously, the motion sensor 124 may be installed on/in or otherwise integral with a HMD 100/300, and may take various forms, such as an accelerometer, gyroscope, magnetometer, gravity sensor, or any combination thereof.

At block 404, the system, e.g., by way of gait inference module 230, may analyze the motion sensor data to infer feature(s) of a gait of the user. For example, at block 406, gait inference module 230 may apply the motion sensor data (raw or preprocessed) as input across a trained machine learning model/classifier to generate output indicative of feature(s) of the user's gait. As noted previously, these gait features may include, but are not limited to, stride length, stride width, walking speed, etc.

At block 408, the system, e.g., by way of cognitive load inference module 234, may infer, e.g., using logic 122 onboard HMD 100/300 or elsewhere, a cognitive load of the user based on the feature(s) of the gait. In some examples, the operation(s) of block 408 may include, at block 410, cognitive load inference module 234 applying the gait feature(s) as input(s), alone or in concert with other non-gait features, across a trained machine learning model (or classifier) to generate output indicative of the user's cognitive load. As noted previously, when a user is walking, some cognitive load inputs such as PPG or galvanic skin response may become noisy, and other cognitive load inputs may become, relatively speaking, more reliable. Accordingly, in some examples, at block 412, a weight applied to another input of the plurality of inputs other than the gait feature(s) may be altered (e.g., increased, reduced, or multiplied by zero) in response to a presence of the feature(s) of the gait,

At block 414, the system, e.g., by way of HMD input module 236 or by another similar module on another computing device, may take various responsive actions based on the inferred cognitive load. For example, application activities and/or notifications may be suppressed so that the user can focus on the task at hand. Objects in a physical environment may be visually annotated and/or emphasized on a display of an AR-style HMD 100/300. A user's cognitive load during training may be monitored and used, for instance, to update the training (e.g., increasing or decreasing the challenge). And so on.

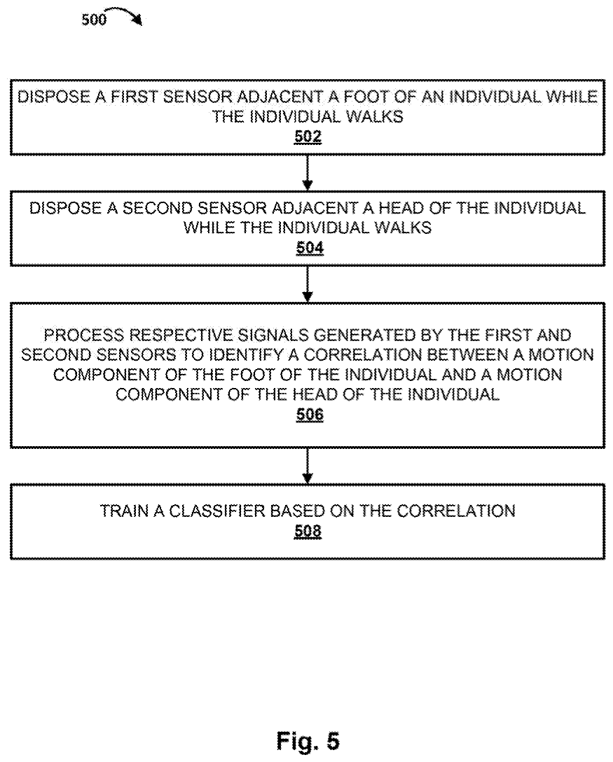

FIG. 5 illustrates a flowchart of an example method 500 for training a classifier/machine learning model that may be used, e.g., by gait inference module 230, to infer a feature of a user's gait. As a consequence of the operations of FIG. 5, the classifier/machine learning model may be trained to map a user's head movement to a feature of the user's gait. Similar operations may be performed to train other machine learning models/classifiers described herein, such as those used by cognitive load inference module 234.

Some operations of FIG. 5 may be performed by a processor, such as a processor of the various computing devices/systems described herein, including logic 122. For convenience, operations of method 500 will be described as being performed by a system configured with selected aspects of the present disclosure. Other implementations may include additional operations than those illustrated in FIG. 5, may perform operations(s) of FIG. 5 in a different order and/or in parallel, and/or may omit various operations of FIG. 5.

At block 502, a first sensor may be disposed adjacent a foot of an individual while the individual walks. For example, the first sensor may be deployed on or within the user's shoe, in their sock, or taped to the user's ankle. In some cases, two such sensors may be deployed, one for each foot of the user.

The first sensor may generate positional data, acceleration data, and/or vibrational data, and may take the form of an accelerometer, gyroscope, magnetometer, gravity meter, piezoelectric sensor, and/or any combination thereof. In some examples, a motion component generated by the first sensor that us used for training may include a vertical displacement of the user's foot. For example, a period of time without a change in a vertical displacement signal generated by the foot-mounted motion sensor (also referred to as a “trough”) may correspond to a planted foot of the individual. In other implementations, a planted foot of the user may be detected as a vibration sensed by a piezoelectric sensor or accelerometer, Additional motion components may also be calculated (e.g., indirectly) from a sensor disposed on the individual's foot, such as a stride length (x component) and stride width (z component).

Because gait is cyclical, any features extracted from the position of the feet that can be converted to a measure of distance can be used to calculate walking speed. Put another way, walking speed can be calculated as distance traversed in the time between any two gait events. For example, in some examples, walking speed is calculated in some examples as stride length divided by time between successive foot plants of the same foot. Alternatively, walking speed can be calculated using step length, which may be calculated based on initial contact of one foot to initial contact of the other foot, divided by the time between those two contacts. In some examples, the peak of the acceleration signal in the x-direction (with respect to the direction of travel) that may occur at the same time during the mid-swing period of gait can be used to derive position, and therefore, displacement of the user's foot over the time between these peaks.

At block 504, a second sensor may be disposed adjacent a head of the individual while the individual walks. This second sensor may share various characteristics with motion sensor 124 described previously, The second sensor may also generate a signal that includes a component that corresponds to vertical displacement, this time of the user's head, rather than their foot.

At block 506, the system may process respective signals generated by the first and second sensors to identify a correlation between a motion component of the foot of the individual and a motion component of the head of the individual. For example, one correlation may be identified between vertical displacement of the user's foot and vertical displacement of the user's head. Additionally or alternatively, another correlation may be found between a walking speed determined from the signal generated by the foot-mounted first sensor and a component of the signal generated by the head-mounted second sensor. At block 508, the system may train the classifier based on and/or to include the correlation.

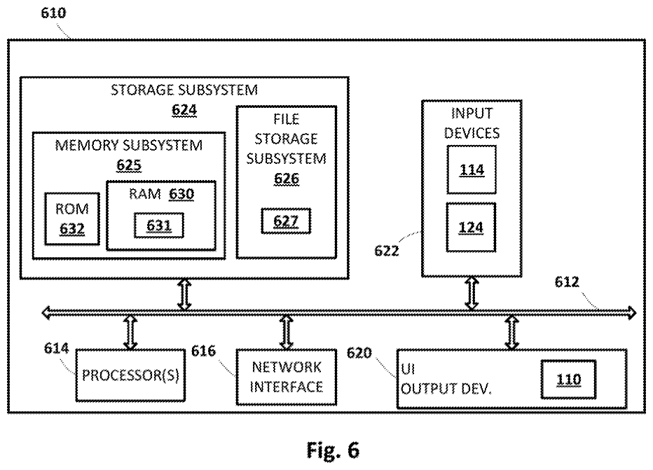

FIG. 6 is a block diagram of an example computer system 610, which in some examples be representative of components found on HMD 100/300 and/or computing devices 350A-D. Computer system 610 may include a processor 614 which communicates with a number of peripheral devices via bus subsystem 612. These peripheral devices may include a storage subsystem 626, including, for example, a memory subsystem 625 and a file storage subsystem 626, user interface output devices 620, input devices 622, and a network interface subsystem 616. The input and output devices allow user interaction with computer system 610. Network interface subsystem 616 provides an interface to outside networks and is coupled to corresponding interface devices in other computer systems.

Input devices 622 may include devices such as a keyboard, pointing devices such as a mouse, trackball, a touch interaction surface, a scanner, a touchscreen incorporated into the display, audio input devices such as voice recognition systems, microphones, vision sensor 114, motion sensor 124, other sensors (e.g., sensors 240-244 in FIG. 2), and/or other types of input devices. In general, use of the term “input device” is intended to include all possible types of devices and ways to input information into computer system 610 or onto a communication network.

User interface output devices 620 may include a display subsystem that includes display 110, a printer, a fax machine, or non-visual displays such as audio output devices. The display subsystem may include a cathode ray tube (“CRT”), a flat-panel device such as a liquid crystal display (“LCD”), a projection device, or some other mechanism for creating a visible image. The display subsystem may also provide non-visual display such as via audio output devices. In general, use of the term “output device” is intended to include all possible types of devices and ways to output information from computer system 610 to the user or to another machine or computer system.

Storage subsystem 624 stores machine-readable instructions and data constructs that provide the functionality of some or all of the modules described herein. These machine-readable instruction modules are generally executed by processor 614 alone or in combination with other processors. Memory 625 used in the storage subsystem 624 may include a number of memories.

For example, a main random access memory (“RAM”) 630 may be used during program execution to store, among other things, instructions 631 for inferring and utilizing gait features as described herein. Memory 625 used in the storage subsystem 624 may also include a read-only memory (“ROM”) 632 in which fixed instructions are stored.

A file storage subsystem 626 may provide persistent or non-volatile storage for program and data files, including instructions 627 for inferring and utilizing gait features as described herein, and may include a hard disk drive, a floppy disk drive along with associated removable media, a CD-ROM drive, an optical drive, or removable media cartridges. The modules implementing the functionality of certain implementations may be stored by file storage subsystem 626 in the storage subsystem 626, or in other machines accessible by the processor(s) 614.

Bus subsystem 612 provides a mechanism for letting the various components and subsystems of computer system 610 communicate with each other as intended. Although bus subsystem 612 is shown schematically as a single bus, other implementations of the bus subsystem may use multiple busses,

Computer system 610 may be of varying types including a workstation, server, computing cluster, blade server, server farm, or any other data processing system or computing device. Due to the ever-changing nature of computers and networks, the description of computer system 610 depicted in FIG. 6 is intended as one non-limited example for purposes of illustrating some implementations. Many other configurations of computer system 610 are possible having more or fewer components than the computer system depicted in FIG. 6.

Although described specifically throughout the entirety of the instant disclosure, representative examples of the present disclosure have utility over a wide range of applications, and the above discussion is not intended and should not be construed to be limiting, but is offered as an illustrative discussion of aspects of the disclosure.

What has been described and illustrated herein is an example of the disclosure along with some of its variations. The terms, descriptions and figures used herein are set forth by way of illustration and are not meant as limitations. Many variations are possible within the scope of the disclosure, which is intended to be defined by the following claims—and their equivalents—in which all terms are meant in their broadest reasonable sense unless otherwise indicated.

What is claimed is:

1. A method for inferring cognitive load of a user, comprising:
generating, with a motion sensor disposed adjacent a head of the user, motion sensor data indicative of head movement of the user;
analyzing, using a processor, the motion sensor data to infer a feature of a gait of the user; and
inferring, using the same processor or a different processor, a cognitive load of the user based on the feature of the gait.

2. The method of claim 1, wherein the motion sensor is integral with or installed in a head-mounted display worn by the user.

3. The method of claim 1, wherein analyzing the motion sensor data to infer the feature of the gait of the user comprises applying the motion sensor data as input across a trained machine learning model to generate output indicative of the feature of the gait.

4. The method of claim 3, wherein the machine learning model is trained to map head movement to the feature of the gait.

5. The method of claim 3, wherein the machine learning model comprises a support vector machine, a random forest, a decision tree, or a neural network.

6. The method of claim 1, wherein the feature of the gait comprises a walking speed of the user or a stride length of the user,

7. The method of claim 1, wherein the inferring comprises applying the feature of the gait as one of a plurality of inputs across a trained machine learning model to generate output indicative of the cognitive load of the user.

8. The method of claim 7, comprising altering a weight applied to another input of the plurality of inputs in response to a presence of the feature of the gait.

9. A head-mounted display (“HMD”) comprising:
a motion sensor to produce a signal indicative of captured motion; and
circuitry operably coupled with the motion sensor, the circuitry to:
process a signal generated by the motion sensor o estimate an attribute of a gait performed by a user wearing the HMD; and
facilitate estimation of a cognitive load of the user based on the attribute of the gait.

10. The HMD of claim 9, wherein to facilitate the estimation, the circuitry is to transmit data indicative of the attribute of the gait to a remote computing device.

11. The HMD of claim 10, wherein the remote computing device comprises a mobile phone, and the data indicative of the attribute is transmitted from the HMD to the mobile phone over a personal area network.

12. The HMD of claim 9, wherein to facilitate the estimation, the circuitry is to analyze the attribute of the gait alongside other inputs to estimate the cognitive load.

13. The HMD of claim 9, wherein the circuitry is to generate, for rendition on a display of the HMD, information about the estimated cognitive load of the user.

14. A non-transitory computer-readable medium comprising instructions that, in response to execution of the instructions by a processor of a head-mounted display (“HMD”), cause the processor to:
receive data indicative of motion of a head of user, wherein the data is based on output of a motion sensor disposed on or within the HMD while the user gaits;
extract a feature of the user's gait from the data indicative of motion of the head of the user; and
infer a cognitive load of the user based on the extracted feature.

15. The non-transitory computer-readable medium of claim 14, further comprising instructions that cause the processor to:
visually emphasize, on a display of the HMD, an object in the user's path based on the inferred cognitive load.
